(19) United States
(12) Patent Application Publication
(10) Pub. No.: US 2004/0176656 A1
(43) Pub. Date: Sep. 9, 2004

(54) Rare earth metals as oxidative dehydrogenation catalysts

(75) Inventors: Joe D. Allison, Ponca City, OK (US); Sriram Ramani, Ponca City, OK (US); Zhen Chen, Ponca City, OK (US); Lisa M. Carmichael, Ponca City, OK (US); Shang Y. Chen, Oklahoma City, OK (US); Steve R. McDonald, Ponca City, OK (US); Anne Gaffney, West Chester, PA (US)

Correspondence Address: DAVID W. WESTPHAL, CONOCOPHILLIPS COMPANY, P.O. BOX 1267, PONCA CITY, OK, 74602-1267, US

(73) Assignee: ConocoPhillips Company, Houston, TX

(21) Appl. No.: 10/806,882

(22) Filed: Mar. 23, 2004

Related U.S. Application Data
(62) Division of application No. 10/266,405, filed on Oct. 8, 2002.

(51) Int. Cl. C07C 5/327; C07C 5/373

(52) U.S. Cl. 585/658

(57) ABSTRACT

Catalysts and methods useful for the production of olefins from alkanes via oxidative dehydrogenation (ODH) are disclosed. The ODH catalysts include a base metal selected from the group consisting of lanthanide metals, their oxides, and combinations thereof. The base metal is more preferably selected from the group consisting of samarium, cerium, praseodymium, terbium, their corresponding oxides and combinations thereof. The base metal loading is preferably between about 0.5 and about 20 weight percent and more preferably between about 2 and about 10 weight percent. Optionally, the ODH catalysts are further comprised of a Group VIII promoter metal present at trace levels. The Group VIII promoter metal is preferably platinum, palladium or a combination thereof and is preferably present at a promoter metal loading of between about 0.005 and about 0.1 weight percent. Optionally, the ODH catalyst is supported on a refractory support.

### CROSS-REFERENCE TO RELATED APPLICATIONS

This application is a divisional of U.S. patent application Ser. No. 10/266,405, filed Oct. 10, 2002 and entitled “Rare Earth Metals as Oxidative-Dehydrogenation Catalysts,” which is incorporated herein by reference.

### STATEMENT REGARDING FEDERALLY SPONSORED RESEARCH OR DEVELOPMENT

Not Applicable.

### TECHNICAL FIELD OF THE INVENTION

This invention relates to catalysts and processes for oxidative dehydrogenation (ODH) of hydrocarbons. More particularly, this invention relates to ODH catalysts comprised of lanthanide metals and to ODH processes that use these ODH catalysts to produce alkenes from alkanes.

### BACKGROUND OF THE INVENTION

There is currently a significant interest in various types of hydrocarbon processing reactions. One such class of reactions involves the chemical conversion of natural gas, a relatively low value reactant, to higher value products. Natural gas comprises several components, including alkanes. Alkanes are saturated hydrocarbons—i.e., compounds consisting of hydrogen (H) and carbon (C)—whose molecules contain carbon atoms linked together by single bonds. The principal alkane in natural gas is methane; however, significant quantities of longer-chain alkanes such as ethane (CH₃CH₃), propane (CH₃CH₂CH₃) and butane (CH₃CH₂CH₂CH₃) are also present. Unlike even longer-chain alkanes, these so-called lower alkanes are gaseous under ambient conditions.

The interest in the chemical conversion of the lower alkanes in natural gas stems from a variety of factors. First, vast reserves of natural gas have been found in remote areas where no local market exists. There is great incentive to exploit these natural gas formations because natural gas is predicted to outlast liquid oil reserves by a significant margin. Unfortunately, though, the transportation costs for the lower alkanes are generally prohibitive, primarily because of the extremely low temperatures needed to liquefy these highly volatile gases for transport. Consequently, there is considerable interest in techniques for converting methane and other gaseous hydrocarbons to higher value, more easily transported, products at the remote site. A second factor driving research into commercial methods for chemical conversion of lower alkanes is their abundant supply at many refineries and the relatively few commercially-viable means of converting them to more valuable products.

Several hydrocarbon processing techniques are currently being investigated for the chemical conversion of lower alkanes. One such technique involves the conversion of methane to higher chain-length alkanes that are liquid or solid at room temperature. This conversion of methane to higher hydrocarbons is typically carried out in two steps. In the first step, methane is partially oxidized to produce a mixture of carbon monoxide and hydrogen known as synthesis gas or syngas. In a second step, the syngas is converted to liquid and solid hydrocarbons using the Fischer-Tropsch process. This method allows the conversion of synthesis gas into liquid hydrocarbon fuels and solid hydrocarbon waxes. The high molecular weight waxes thus produced provide an ideal feedstock for hydrocracking, which ultimately yields high quality jet fuel and superior high decane value diesel fuel blending components.

Another important class of hydrocarbon processing reactions are dehydrogenation reactions. In a dehydrogenation process, alkanes can be dehydrogenated to produce alkenes. Alkenes, also commonly called olefins, are unsaturated hydrocarbons whose molecules contain one or more pairs of carbon atoms linked together by a double bond. Generally, olefin molecules are represented by the chemical formula R′CH═CHR, where C is a carbon atom, H is a hydrogen atom, and R and R′ are each an atom or a pendant molecular group of varying composition. One example of a dehydrogenation reaction is the conversion of ethane to ethylene [1]:

C₂H₆+Heat→C₂H₄+H₂ [1]

The non-oxidative dehydrogenation of ethane to ethylene is endothermic, meaning that heat energy must be supplied to drive the reaction.

Olefins containing two to four carbon atoms per molecule—i.e., ethylene, propylene, butylene and isobutylene—are gaseous at ambient temperature and pressure. In contrast, those containing five or more carbon atoms are usually liquid under ambient conditions. More importantly, alkenes also are higher value chemicals than their corresponding alkanes. This is true, in part, because alkenes are important feedstocks for producing various commercially useful materials such as detergents, high-octane gasolines, pharmaceutical products, plastics, synthetic rubbers and viscosity additives. Ethylene, a raw material in the production of polyethylene, is the one of the most abundantly produced chemicals in the United States and cost-effective methods for producing ethylene are of great commercial interest.

Traditionally, the dehydrogenation of hydrocarbons has been carried out using fluid catalytic cracking (FCC), a non-oxidative dehydrogenation process, or steam cracking. Heavy alkenes, those containing five or more carbon atoms, are typically produced by FCC; in contrast, light olefins, those containing two to four carbon atoms, are typically produced by steam cracking. FCC and steam cracking have several drawbacks. First, both processes are highly endothermic requiring input of energy. In addition, a significant amount of the alkane reactant is lost as carbon deposits known as coke. These carbon deposits not only decrease yields but also deactivate the catalysts used in the FCC process. The costs associated with heating, yield loss and catalyst regeneration render these processes expensive even without regard to catalyst costs.

Recently, there has been increased interest in oxidative dehydrogenation (ODH) as an alternative to FCC and steam cracking. In ODH, alkanes are dehydrogenated in the presence of an oxidant such as oxygen, typically in a short contact time reactor containing an ODH catalyst. ODH can be used, for example, to convert ethane and oxygen to ethylene and water [2]:

C₂H_{6 +1/2}O₂→C₂H₄+H₂O+Heat [2]

Thus, ODH provides an alternative chemical route to generating alkenes from alkanes. Unlike non-oxidative dehydrogenation, however, ODH is exothermic, meaning that it produces rather than requires heat energy.

Although ODH involves the use of a catalyst, which is referred to herein as an ODH catalyst, and is therefore literally a catalytic dehydrogenation, ODH is distinct from what is normally called “catalytic dehydrogenation” in that the former involves the use of an oxidant and the latter does not. ODH is attractive because the capital costs for olefin production via ODH are significantly less than with the traditional processes. ODH, unlike traditional FCC and steam cracking, uses simple fixed bed reactor designs and high volume throughput.

More important, however, is the fact that ODH is exothermic. The net ODH reaction can be viewed as two separate processes: an endothermic dehydrogenation of an alkane coupled with a strongly exothermic combustion of hydrogen, as depicted in [3]:

$$\frac{2 {H}_{6} + Heat -> {C}_{2} {H}_{4} + {H}_{2} \\ 1 \text{/} 2 {O}_{2} + {H}_{2} -> {H}_{2} O + Heat}{{C}_{2} {H}_{6} + 1 \text{/} 2 {O}_{2} -> {C}_{2} {H}_{4} + {H}_{2} O + Heat} \qquad [ 3 ]$$

Energy savings over traditional, endothermic processes can be especially significant if the heat produced in the ODH process is recaptured and recycled.

Catalysis plays a central role in a number of hydrocarbon processing techniques including dehydrogenation reactions. Each of these methods shares a common attribute: successful commercial scale operation for catalytic hydrocarbon processing depends upon high hydrocarbon feedstock conversion at high throughput and with high selectivity for the desired reaction products. In each case, the yields and selectivities of catalytic hydrocarbon processing are affected by several factors. One of the most important of these factors is the choice of catalyst composition, which significantly affects not only the yields and product distributions but also the overall economics of the process. Unfortunately, few catalysts offer both the performance and cost necessary for large-scale industrial use.

Catalyst cost is one of the most significant economic considerations in ODH processes. Non-oxidative dehydrogenation reactions frequently employ relatively inexpensive iron-oxide based catalysts. In contrast, ODH catalysts typically utilize relatively expensive precious metals—e.g., platinum—as promoters that assist in the combustion reaction. Despite various attempts, large quantities of catalyst are frequently lost during ODH processing, including the expensive promoter metal component. Because promoter metals frequently account for the majority of the catalyst cost, a major cost for ODH is the cost of replenishing lost promoter metal.

Despite a vast amount of research effort in this field, there is still a great need to identify effective but low-cost ODH catalyst systems for olefin synthesis, so as to maximize the value of the olefins produced and thus optimize the process economics. In addition, to ensure successful operation on a commercial scale, the ODH process must be able to achieve a high conversion of the hydrocarbon feedstock at high gas hourly space velocities, while maintaining high selectivity of the process to the desired products.

### SUMMARY OF THE INVENTION

The preferred embodiments of the present invention include ODH catalysts that comprise one or more base metals, metal oxides, or mixed metal/metal oxides. The base metal is selected from the group consisting of lanthanide metals, their oxides and combinations thereof. More preferably, the base metal is selected from the group consisting of samarium, cerium, praseodymium, terbium, their corresponding oxides and combinations thereof. The base metal is preferably present at a base metal loading of between about 0.5 and about 20 weight percent of the ODH catalyst, more preferably between about 1 and about 12, and still more preferably between about 2 and about 10 weight percent.

Some of the preferred embodiments of the present invention include ODH catalysts further comprised of one or more promoter metals. When present, the promoter metal is a Group VIII metal, preferably rhodium, platinum, palladium, ruthenium or iridium or a combination thereof. The promoter metal is preferably present at a promoter metal loading of between about 0.005 and about 0.1 weight percent of the ODH catalyst, more preferably between about 0.005 and about 0.095, still more preferably between about 0.005 and about 0.075, and yet still more preferably between about 0.005 and about 0.05 weight percent. The molar ratio of the base metal to the optional promoter metal is preferably about 10 or higher, more preferably about 15 or higher, still more preferably about 20 or higher, and yet still more preferably about 25 or higher.

Optionally, the ODH catalyst may comprise a refractory support. Preferably, the refractory support is selected from the group consisting of zirconia, magnesium stabilized zirconia, zirconia stabilized alumina, yttrium stabilized zirconia, calcium stabilized zirconia, alumina, cordierite, titania, silica, magnesia, niobia, vanadia, nitrides, silicon nitride, cordierite, cordierite-alpha alumina, zircon mullite, spodumene, alumina-silica magnesia, zircon silicate, sillimanite, magnesium silicates, zircin, petalite, carbon black, calcium oxide, barium sulfate, silica-alumina, alumina-zirconia, alumina-chromia, alumina-ceria, and combinations thereof. More preferably, the refractory support comprises alumina, zirconia, stabilized aluminas, stabilized zirconias or combinations thereof.

The preferred embodiments of the present invention also include methods for performing ODH processes that employ the ODH catalysts disclosed herein. Preferably, the ODH process is performed in a short-contact time reactor (SCTR). The reactant mixtures for the preferred embodiments of the present invention comprise hydrocarbons, preferably alkanes, and an oxidant, preferably a molecular oxygen-containing gas. According to some preferred embodiments, the composition of the reactant mixture is such that the atomic oxygen-to-carbon ratio is between about 0.05:1 and about 5:1. Preferably, the ODH catalyst composition and the reactant mixture composition are such that oxidative dehydrogenation promoting conditions can be maintained with a preheat temperature of about 600° C. or less. More preferably, the ODH catalyst composition and the reactant mixture composition are such that oxidative dehydrogenation promoting conditions can be maintained with a preheat temperature of about 300° C. or less. According to some preferred embodiments, the ODH processes operate at a gas-hourly space velocity of between about 20,000 and about ₂00,000,000 hr⁻¹ and at a temperature of between about 600° C. and about 1200° C.

The preferred embodiments of the present invention also include alkenes produced from alkanes using the ODH catalysts and according to the methods described.

### DETAILED DESCRIPTION OF PREFERRED EMBODIMENTS

The preferred embodiments of the present invention derive partly from the discovery that ODH catalysts comprised of lanthanide metals can provide both high alkane conversion and alkene selectivity, even under high throughput conditions. The preferred embodiments also derive partly from the discovery that trace levels of Group VIII metals in the ODH catalyst can reduce the feedstock pre-heat temperature necessary to initiate and sustain the ODH process. As used herein, the term “ODH catalyst” refers to the overall catalyst including, but not limited to, any base metal, promoter metal and refractory support.

The preferred embodiments of the present invention employ one or more base metals in the ODH catalyst. A variety of base metals exhibit catalytic activity in ODH processes and are within the scope of the present invention. Without limiting the scope of the invention, base metals useful in the preferred embodiments of the present invention include lanthanide metals, their oxides and combinations thereof. More preferably, the base metal is selected from the group consisting of samarium, cerium, praseodymium, terbium, their corresponding oxides and combinations thereof. A combination of base metals is within the scope of the invention. Consequently, references herein to the base metal are not intended to limit the invention to one base metal.

As used herein, the term “base metal loading” refers to the percent by weight base metal in the ODH catalyst, measured as the weight of reduced base metal relative to the overall weight of the ODH catalyst. When present, the base metal is preferably present at a base metal loading of between about 0.5 and about 20 weight percent, more preferably between about 1 and about 12 weight percent, and still more preferably between about 2 and about 10 weight percent.

Some of the preferred embodiments of the present invention include ODH catalysts further comprised of one or more promoter metals. When present, the promoter metal is selected from the group consisting of Group VIII metals—i.e., platinum, rhodium, ruthenium, iridium, nickel, palladium, iron, cobalt and osmium. Rhodium, platinum, palladium, ruthenium, iridium and combinations thereof are preferred promoter metals. However, as is evident to those of skill in the art, other promoter metals can also be used. Furthermore, a combination of promoter metals is also within the scope of the invention. Consequently, references herein to the promoter metal are not intended to limit the invention to one promoter metal.

As used herein, the term “promoter metal loading” refers to the percent by weight promoter metal in the ODH catalyst, measured as the weight of reduced promoter metal relative to the overall weight of the ODH catalyst. Preferably, the promoter metal loading is between about 0.005 and about 0.1 weight percent. The promoter metal loading is more preferably between about 0.005 and about 0.095, still more preferably between about 0.005 and about 0.075, and yet still more preferably between about 0.005 and about 0.05 weight percent. Preferably, the molar ratio of the base metal to the optional promoter metal, when present, is about 10 or higher, more preferably about 15 or higher, still more preferably about 20 or higher, and yet still more preferably about 25 or higher.

Preferably, the base metal and the promoter metal, if present, are deposited on refractory supports configured as wire gauzes, porous monoliths, or particles. The term “monolith” refers to any singular piece of material of continuous manufacture such as solid pieces of metal or metal oxide or foam materials or honeycomb structures. Two or more such catalyst monoliths may be stacked in the catalyst zone of the reactor if desired. For example, the catalyst can be structured as, or supported on, a refractory oxide “honeycomb” straight channel extrudate or monolith, made of cordierite or mullite, or other configuration having longitudinal channels or passageways permitting high space velocities with a minimal pressure drop. Such configurations are known in the art and described, for example, in Structured Catalysts and Reactors, A. Cybulski and J. A. Moulijn (Eds.), Marcel Dekker, Inc., 1998, p. 599-615 (Ch. 21, X. Xu and J. A. Moulijn, “Transformation of a Structured Carrier into Structured Catalyst”), which is hereby incorporated herein by reference.

Some preferred monolithic supports include partially stabilized zirconia (PSZ) foam (stabilized with Mg, Ca or Y), or foams of α-alumina, cordierite, titania, mullite, Zr-stabilized α-alumina, or mixtures thereof A preferred laboratory-scale ceramic monolith support is a porous alumina foam with approximately 6,400 channels per square inch (80 pores per linear inch). Preferred foams for use in the preparation of the catalyst include those having from 30 to 150 pores per inch (12 to 60 pores per centimeter). The monolith can be cylindrical overall, with a diameter corresponding to the inside diameter of the reactor tube.

Alternatively, other refractory foam and non-foam monoliths may serve as satisfactory supports. The promoter metal precursor and any base metal precursor, with or without a ceramic oxide support forming component, may be extruded to prepare a three-dimensional form or structure such as a honeycomb, foam or other suitable tortuous-path structure.

More preferred catalyst geometries employ distinct or discrete particles. The terms “distinct” or “discrete” particles, as used herein, refer to supports in the form of divided materials such as granules, beads, pills, pellets, cylinders, trilobes, extrudates, spheres, other rounded shapes or another manufactured configuration. Alternatively, the divided material may be in the form of irregularly shaped particles. Preferably at least a majority—i.e., greater than about 50 percent—of the particles or distinct structures have a maximum characteristic length (i.e., longest dimension) of less than six millimeters, preferably less than three millimeters. Preferably, these particulate-supported catalysts are prepared by impregnating or washcoating the promoter metal and base metal, if present, onto the refractory particulate support.

Numerous refractory materials may be used as supports in the present invention. Without limiting the scope of the invention, suitable refractory support materials include zirconia, magnesium stabilized zirconia, zirconia stabilized alumina, yttrium stabilized zirconia, calcium stabilized zirconia, alumina, cordierite, titania, silica, magnesia, niobia, vanadia, nitrides, silicon nitride, cordierite, cordierite-alpha alumina, zircon mullite, spodumene, alumina-silica magnesia, zircon silicate, sillimanite, magnesium silicates, zircin, petalite, carbon black, calcium oxide, barium sulfate, silica-alumina, alumina-zirconia, alumina-chromia, alumina-ceria, and combinations thereof. Preferably, the refractory support comprises alumina, zirconia, stabilized aluminas, stabilized zirconias or combinations thereof. Alumina is preferably in the form of alpha-alumina (α-alumina); however, the other forms of alumina have also demonstrated satisfactory performance.

The base metal and promoter metal, when present, may be deposited in or on the refractory support by any method known in the art. Without limiting the scope of the invention, acceptable methods include incipient wetness impregnation, chemical vapor deposition, co-precipitation, and the like. Preferably, the base and promoter metals are deposited by the incipient wetness technique.

The preferred embodiments of the processes of the present invention employ a hydrocarbon feedstock and an oxidant feedstock that are mixed to yield a reactant mixture, which is sometimes referred to herein as the reactant gas mixture. Preferably, the hydrocarbon feedstock comprises one or more alkanes having between two and ten carbon atoms. More preferably, the hydrocarbon feedstock comprises one or more alkanes having between two and five carbon atoms. Without limiting the scope of the invention, representative examples of acceptable alkanes are ethane, propane, butane, isobutane and pentane. The hydrocarbon feedstock preferably comprises ethane.

The oxidant feedstock comprises an oxidant capable of oxidizing at least a portion of the hydrocarbon feedstock. Appropriate oxidants may include, but are not limited to, I₂, O₂, N₂O, CO₂ and SO₂. Use of the oxidant shifts the equilibrium of the dehydrogenation reaction toward complete conversion through the formation of compounds containing the abstracted hydrogen (e.g., H₂O, HI and H₂S). Preferably, the oxidant comprises a molecular oxygen-containing gas. Without limiting the scope of the invention, representative examples of acceptable molecular oxygen-containing gas feedstocks include pure oxygen gas, air and O₂-enriched air.

As depicted in equation [4], the complete combustion of an alkane requires a stoichiometrically predictable quantity of oxygen:

CₙH_{2n°2}+[(3n+1)/2]O₂→nCO₂+[n+1]H₂O [4

According to equation 4, an atomic oxygen-to-carbon ratio of 3n+1:n represents the stoichiometric ratio for complete combustion where n equals the number of carbons in the alkane. For alkanes with between 2 and 10 carbon atoms, the stoichiometric ratio of oxygen atoms to carbon atoms for complete combustion ranges between 3.5:1 and 3.1:1. Preferably, the composition of the reactant mixture is such that the atomic oxygen-to-carbon ratio is between about 0.05:1 and about 5:1. In some embodiments, the reactant mixture may also comprise steam. Steam may be used to activate the catalyst, remove coke from the catalyst, or serve as a diluent for temperature control. The ratio of steam to carbon by weight, when steam is added, may preferably range from about 0 to about 1.

Preferably, a short contact time reactor (SCTR) is used. Use of a SCTR for the commercial scale conversion of light alkanes to corresponding alkenes allows reduced capital investment and increases alkene production significantly. The preferred embodiments of the present invention employ a very fast contact (i.e., millisecond range)/fast quench (i.e., less than one second) reactor assembly such as those described in the literature. For example, co-owned U.S. Pat. Nos. 6,409,940 and 6,402,898 describe the use of a millisecond contact time reactor for use in the production of synthesis gas by catalytic partial oxidation of methane. The disclosures of these references are hereby incorporated herein by reference.

The ODH catalyst may be configured in the reactor in any arrangement including fixed bed, fluidized bed, or ebulliating bed (sometimes referred to as ebullating bed) arrangements. A fixed bed arrangement employs a stationary catalyst and a well-defined reaction volume whereas a fluidized bed utilizes mobile catalyst particles. Conventional fluidized beds include bubbling beds, turbulent fluidized beds, fast fluidized beds, concurrent pneumatic transport beds, and the like. A fluidized bed reactor system has the advantage of allowing continuous removal of catalyst from the reaction zone, with the withdrawn catalyst being replaced by fresh or regenerated catalyst. A disadvantage of fluidized beds is the necessity of downstream separation equipment to recover entrained catalyst particles. Preferably, the catalyst is retained in a fixed bed reaction regime in which the-catalyst is retained within a well-defined reaction zone. Fixed bed reaction techniques are well known and have been described in the literature. Irrespective of catalyst arrangement, the reactant mixture is contacted with the catalyst in a reaction zone while maintaining reaction promoting conditions.

The reactant gas mixture is heated prior to or as it passes over the catalyst such that the reaction initiates. In accordance with one preferred embodiment of the present invention, a method for the production of olefins includes contacting a pre-heated alkane and a molecular-oxygen containing gas with a catalyst containing a lanthanide base metal and a refractory support sufficient to initiate the oxidative dehydrogenation of the alkane, maintaining a contact time of the alkane with the catalyst for less than 200 milliseconds, and maintaining oxidative dehydrogenation promoting conditions. Preferably, the ODH catalyst composition and the reactant mixture composition are such that oxidative dehydrogenation promoting conditions can be maintained with a preheat temperature of about 600° C. or less. More preferably, the ODH catalyst composition and the reactant mixture composition are such that oxidative dehydrogenation promoting conditions can be maintained with a pre-heat temperature of about 300° C. or less.

Reaction productivity, conversion and selectivity are affected by a variety of processing conditions including temperature, pressure, gas hourly space velocity (GHSV) and catalyst arrangement within the reactor. As used herein, the term “maintaining reaction promoting conditions” refers to controlling these reaction parameters, as well as reactant mixture composition and catalyst composition, in a manner in which the desired ODH process is favored.

The reactant mixture may be passed over the catalyst in any of a wide range of gas hourly space velocities. Gas hourly space velocity (GHSV) is defined as the volume of reactant gas per volume of catalyst per unit time. Although for ease in comparison with prior art systems space velocities at standard conditions have been used to describe the present invention, it is well recognized in the art that residence time is inversely related to space velocity and that high space velocities correspond to low residence times on the catalyst and vice versa. High throughput systems typically employ high GHSV and low residence times on the catalyst.

Preferably, GHSV for the present process, stated as normal liters of gas per liters of catalyst per hour, ranges from about 20,000 to about 200,000,000 hr⁻¹, more preferably from about 50,000 to about 50,000,000 hr⁻¹, and most preferably from about 100,000 to about 3,000,000 hr⁻¹. The GHSV is preferably controlled so as to maintain a reactor residence time of no more than about 200 milliseconds for the reactant mixture. An effluent stream of product gases including alkenes, unconverted alkanes, H₂O and possibly CO, CO₂, H₂ and other by-products exits the reactor. In a preferred embodiment, the alkane conversion is at least about 40 percent and the alkene selectivity is at least about 30 percent. More preferably, the alkane conversion is at least about 60 percent and the alkene selectivity is at least about 50 percent. Still more preferably, the alkane conversion is at least about 80 percent and the alkene selectivity is at least about 55 percent. Still yet more preferably, the alkane conversion is at least about 85 percent and the alkene selectivity is at least about 60 percent.

Hydrocarbon processing techniques typically employ elevated temperatures to achieve reaction promoting conditions. According to some preferred embodiments of the present invention, the step of maintaining reaction promoting conditions includes pre-heating the reactant mixture to a temperature between about 30° C. and about 750° C., more preferably not more than about 600° C. The ODH process typically occurs at temperatures of from about 450° C. to about 2,000° C., more preferably from about 700° C. to about 1,200° C. As used herein, the terms “autothermal,” “adiabatic” and “self-sustaining” mean that after initiation of the hydrocarbon processing reaction, additional or external heat need not be supplied to the catalyst in order for the production of reaction products to continue. Under autothermal or self-sustaining reaction conditions, exothermic reactions provide the heat for endothermic reactions, if any. Consequently, under autothermal process conditions, an external heat source is generally not required.

Hydrocarbon processing techniques frequently employ atmospheric or above atmospheric pressures to maintain reaction promoting conditions. Some embodiments of the present invention entail maintaining the reactant gas mixture at atmospheric or near-atmospheric pressures of approximately 1 atmosphere while contacting the catalyst. Advantageously, certain preferred embodiments of the process are operated at above atmospheric pressure to maintain reaction promoting conditions. Some preferred embodiments of the present invention employ pressures up to about 32,000 kPa (about 320 atmospheres), more preferably between about 200 and about 10,000 kPa (between about 2 and about 100 atmospheres).

### EXAMPLES

The following examples demonstrate the effect of various catalyst compositions on the ODH process. The refractory support material, alumina, was purchased from Porvair Advanced Materials. In some experiments, the alumina was utilized without the addition of any base or promoter metal. In other experiments, a base and/or promoter metal were added to the refractory support by incipient wetness, a deposition technique well-known in the art. The soluble metal salts employed for incipient wetness were nitrates, acetates, chlorides, acetylacetonates or the like. The base metal was added first and comprised one of the lanthanide metals. After the base metal was applied, the catalyst was dried at 80° C. for 1 hour followed by calcination in air at 500° C. for 3 hours. The promoter metal, when added, comprised either rhodium, iridium or ruthenium and was added using the same procedures as for the base metals. The finished catalyst was then reduced in 50 percent hydrogen in nitrogen at 500° C. for 3 hours. In each case, the refractory support was a monolith.

The effects of promoter metal loading and base metal loading on alkane conversion, alkene selectivity and alkene yield for a variety of catalyst compositions employing alumina refractory supports are shown in Table 1. In addition, Table 1 depicts the gas preheat temperature necessary to initiate the reaction for each catalyst. The reactant gas mixture comprised O₂ and ethane, and the molar ethane-to-O₂ ratio of the feed was 2.0 (or an atomic ratio C/O of 2.0) with a total reactant gas mixture flow rate of 3 standard liters per minute. The reactor pressure was about from 4 to 5 psig (128.9 to 135.8 kPa).

As depicted in Table 1, the cerium- and lanthanum-based catalysts failed to light off under the experimental conditions employed. Although the bare alumina and praseodymium-based catalysts did light off, neither ODH catalyst allowed for a sustained dehydrogenation reaction. In contrast, however, ODH catalysts comprised of terbium and samarium provided for sustained dehydrogenation reactions. In particular, the terbium-based catalyst gave unexpectedly good results. Not only was the terbium-based ODH catalyst active using a preheat temperature of 300° C., but it gave the best conversion, selectivity and yield results of the lanthanide metals tested.

TABLE 1
Results from Lanthanide Metals ODH Catalysts

| Catalyst | Required | Ethane | Ethylene | Ethylene | |
|---|---|---|---|---|---|
| Composition | Preheat | Conversion | Selectivity | Yield | |
| Weight % | (° C.) | (Mole Percent) | (Mole Percent) | (Percent) | Comment |
| 7.0 Ce/Al₂O₃ | | | | | failed to light off |
| 6.9 La/Al₂O₃ | | | | | failed to light off |
| 7.0 Pr/Al₂O₃ | 350 | 82.2 | 62.9 | 51.7 | reaction not |
| | | | | | sustained |
| 7.9 Tb/Al₂O₃ | 300 | 88.2 | 65.2 | 57.5 | |
| 5.4 Sm/Al₂O₃ | 525 | 81.7 | 56.8 | 46.4 | |
| Al₂O₃ | | | | | reaction not |
| | | | | | sustained |

Weight percents equal to 0.50 mmole metal, except for Sm which is 0.36 mmole

To test the effect of a Group VIII metal on the lanthanide-based ODH catalysts, a rhodium-based alumina ODH catalyst was compared to a variety of rhodium/lanthanide alumina ODH catalysts. Again, the testing conditions employed a molar ethane-to-O₂ ratio in the reactant gas mixture of 2.0 (or an atomic ratio C/O of 2.0) with a total flow rate of 3 standard liters per minute. The reactor pressure was again about from 4 to 5 psig (128.9 to 135.8 kPa). The results are shown in Table 2.

TABLE 2
Results for 0.01 Weight Percent Rhodium-Promoted
Lanthanide Metal Catalysts

| | | Ethane | Ethylene | | |
|---|---|---|---|---|---|
| Catalyst | Required | Conversion | Selectivity | Ethylene | |
| Composition | Preheat | (Mole | (Mole | Yield | |
| Weight % | (° C.) | Percent) | Percent) | (Percent) | Ln/Rh Ratio |
| 0.01 Rh/7.0 Ce/Al₂O₃ | 300 | 84.3 | 62.1 | 52.3 | 515 |
| 0.01 Rh/La/Al₂O₃^{a} | | | | | 515 |
| 0.01 Rh/7.0 Pr/Al₂O₃ | 300 | 87.7 | 65.0 | 57.0 | 515 |
| 0.01 Rh/7.6 Eu/Al₂O₃^{b} | 400 | | | | 515 |
| 0.01 Rh/8.4 Tm/Al₂O₃ | 300 | 71.1 | 62.0 | 44.0 | 515 |
| 0.01 Rh/7.9 Tb/Al₂O₃ | 300 | 89.3 | 65.1 | 58.1 | 515 |
| 0.01 Rh/8.1 Dy/Al₂O₃ | 300 | 61.3 | 62.1 | 38.1 | 515 |
| 0.01 Rh/8.2 Ho/Al₂O₃ | 300 | 34.4 | 56.4 | 19.4 | 515 |
| 0.01 Rh/8.3 Er/Al₂O₃ | 300 | 71.7 | 62.8 | 45 | 515 |
| 0.01 Rh/8.6 Yb/Al₂O₃ | 300 | 60.2 | 64.3 | 38.7 | 515 |
| 0.01 Rh/8.7 Lu/Al₂O₃ | 300 | 74.4 | 63.8 | 47.5 | 515 |
| 0.01 Rh/Al₂O₃ | 300 | 68.1 | 59.3 | 40.4 | |

^{a}no reaction;
^{b}fuel/O₂ of 2.0 not achieved
Weight percent lanthanides equal to 0.5 mmole; 0.01 weight % Rh = 1.0 × 10⁻³ mmole.

As is evident from Table 2, the rhodium ODH catalyst having no lanthanide (“the rhodium control”) provided an ethane conversion of 68.1 mole percent, an ethylene selectivity of 59.3 mole percent, and an ethylene yield of 40.4 percent. As a group, the lanthanum-based catalysts exhibited a wide range of performance. The lanthanum-based catalyst gave no reaction under the testing conditions while the europium-based catalyst was not operable under the experimental test conditions. The ODH catalysts comprised of dysprosium, holmium and ytterbium performed more poorly than the rhodium control. In fact, they generated poorer results in all categories except that the dysprosium-based ODH catalyst offered marginally better ethylene selectivity than the rhodium control.

ODH catalysts comprised of thulium, erbium and lutetium were only marginally better than the rhodium control. These three averaged an ethane conversion of 72.4 mole percent (as compared with 68.1 mole percent for the rhodium control), an ethylene selectivity of 62.9 mole percent (as compared with 59.3 mole percent for the rhodium control), and an ethylene yield of 45.5 percent (as compared with 40.4 mole percent for the rhodium control). Although the results are better in each category than those achieved with the rhodium control, they do not represent a marked improvement.

In contrast to the other lanthanide-based ODH catalysts in Table 2, the ODH catalysts comprised of cerium, praseodymium, and terbium exhibited markedly improved performance over the rhodium control. These ODH catalysts averaged an ethane conversion of 87.1 mole percent (as compared with 68.1 mole percent for the rhodium control), an ethylene selectivity of 64.1 mole percent (as compared with 59.3 mole percent for the rhodium control), and an ethylene yield of 55.8 percent (as compared with 40.4 mole percent for the rhodium control).

To test the effect of different Group VIII promoter metals, praseodymium-based ODH catalysts were tested with rhodium, ruthenium and iridium promoters. Again, the testing conditions employed a molar ethane-to-O₂ ratio of the reactant gas mixture of 2.0 (or an atomic ratio C/O of 2.0) with a total flow rate of 3 standard liters per minute. The reactor pressure was again about from 4 to 5 psig (128.9 to 135.8 kPa). The results are shown in Table 3.

TABLE 3
Results from Praseodymium ODH Catalysts
Promoted with 0.01 Weight Percent Group 8 Metals

| | Required | Ethane | Ethylene | Ethylene | |
|---|---|---|---|---|---|
| Catalyst Composition | Preheat | Conversion | Selectivity | Yield | Pr/PM |
| Weight % | (° C.) | (Mole Percent) | (Mole Percent) | (Percent) | Ratio |
| 0.01 Rh/7.0 Pr/Al₂O₃ | 300 | 87.7 | 65.0 | 57.0 | 515 |
| 0.01 Ru/7.0 Pr/Al₂O₃ | 300 | 86.5 | 63.1 | 54.6 | 515 |
| 0.01 Ir/7.0 Pr/Al₂O₃ | 300 | 78.2 | 54.4 | 42.6 | 962 |

7.0% Pr = 0.5 mmole;
0.01% Rh, Ru = 1.0 × 10⁻³ mmole,
0.01% Ir = 5.2 × 10⁻⁴ mmole
PM = promoter metal

Unlike the unpromoted praseodymium ODH catalyst in Table 1 that was unable to sustain the dehydrogenation reaction, the promoted praseodymium ODH catalysts tested for Table 3 are highly active. In each case, the catalyst is not only capable of sustaining the dehydrogenation reaction, but also of initiating the reaction using a preheat temperature of 300° C. Based upon the ethane conversion, ethylene selectivity and ethylene yield, both the rhodium- and ruthenium-promoted catalysts performed better than the iridium-promoted catalysts. The following commonly assigned copending application is hereby incorporated herein by reference: “Oxidative Dehydrogenation of Hydrocarbons Using Catalysts With Trace Promoter Metal Loading,” application Ser. No. 10/266,404 filed Oct. 8, 2002. Should the disclosure of any of the patents, patent applications, and publications that are incorporated herein conflict with the present specification to the extent that it might render a term unclear, the present specification shall take precedence.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention.

Accordingly, the scope of protection is not limited by the description set out above, but is only limited by the claims which follow, that scope including all equivalents of the subject matter of the claims. Each and every claim is incorporated into the specification as an embodiment of the present invention. Thus the claims are a further description and are an addition to the preferred embodiments of the present invention. Use of the term “optionally” with respect to any element of a claim is intended to mean that the subject element is required, or alternatively, is not required. Both alternatives are intended to be within the scope of the claim. The discussion of a reference in the Description of Related Art is not an admission that it is prior art to the present invention, especially any reference that may have a publication date after the priority date of this application. The disclosures of all patents, patent applications and publications cited herein are hereby incorporated herein by reference, to the extent that they provide exemplary, procedural or other details supplementary to those set forth herein.

What is claimed is:

1. A method for oxidative dehydrogenation comprising
a) providing a reactant mixture comprising one or more hydrocarbons and an oxidant;
b) providing an ODH catalyst comprising a base metal selected from the group consisting of lanthanide metals, their oxides and combinations thereof;
c) exposing the reactant mixture to the ODH catalyst in a reactor under reaction promoting conditions; and
d) oxidatively dehydrogenating at least a fraction of the one or more hydrocarbons in the reactant mixture.

2. The method of claim 1 wherein the reactor is a short contact time reactor operated at a GHSV between about 20,000 hr⁻¹ and about 200,000,000 hr⁻¹.

3. The method of claim 1 wherein the reactor is a short contact time reactor operated at a GHSV between about 50,000 hr⁻¹ and about 50,000,000 hr⁻¹.

4. The method of claim 1 wherein the oxidant comprises a molecular oxygen-containing gas and the one or more hydrocarbons comprise one or more alkanes.

5. The method of claim 4 wherein the one or more alkanes comprise one or more paraffins with between 2 and 10 carbon atoms.

6. The method of claim 4 wherein the one or more alkanes comprise one or more paraffins with between 2 and 5 carbon atoms.

7. The method of claim 4 further comprising the step of pre-heating the reactant mixture to about 600° C. or less.

8. The method of claim 4 further comprising the step of preheating the reactant mixture to about 300° C. or less.

9. The method of claim 4 wherein the atomic oxygen-to-carbon ratio is between about 0.05:1 and about 5:1.

10. The method of claim 4 wherein the alkane conversion is at least about 40 percent and the alkene selectivity is at least about 35 percent.

11. The method of claim 4 wherein the alkane conversion is at least about 85 percent and the alkene selectivity is at least about 60 percent.

12. The method of claim 1 wherein the base metal is present at a base metal loading between about 0.5 and about 20 weight percent.

13. The method of claim 1 wherein the base metal is present at a base metal loading between about 2 and about 10 weight percent.

14. The method of claim 1 wherein the base metal is selected from the group consisting of samarium, cerium, praseodymium, terbium, their corresponding oxides and combinations thereof.

15. The method of claim 1 wherein the ODH catalyst further comprises a promoter metal selected from the group consisting of Group VIII metals, their oxides and combinations thereof and present at a promoter metal loading between about 0.005 and 0.10 weight percent.

16. The method of claim 1 wherein the ODH catalyst further comprises a promoter metal selected from the group consisting of rhodium, platinum, palladium, ruthenium or iridium and combinations thereof.

17. The method of claim 15 wherein the ODH catalyst has a molar ratio of base metal to promoter metal of about 10 or more.

18. The method of claim 15 wherein the ODH catalyst has a molar ratio of base metal to promoter metal of about 25 or more.

19. The method of claim 1 wherein the ODH catalyst further comprises a refractory support.

20. The method of claim 19 wherein the refractory support is comprised of a material selected from group consisting of zirconia, stabilized zirconias, alumina, stabilized aluminas, and combinations thereof.

21. The method of claim 19 wherein the ODH catalyst further comprises a promoter metal selected from the group consisting of Group VIII metals, their oxides and combinations thereof and present at a promoter metal loading between about 0.005 and 0.10 weight percent.

22. The method of claim 19 wherein the ODH catalyst further comprises a promoter metal selected from the group consisting of rhodium, platinum, palladium, ruthenium or iridium and combinations thereof.

23. The method of claim 21 wherein the ODH catalyst has a molar ratio of base metal to promoter metal of about 10 or more.

24. The method of claim 21 wherein the ODH catalyst has a molar ratio of base metal to promoter metal of about 25 or more.
